(19) United States
(12) Patent Application Publication
(10) Pub. No.: US 2008/0166420 A1
(43) Pub. Date: Jul. 10, 2008

(54) Krill Oil Compositions

(76) Inventor: Scott F. Sones, Niland, CA (US)

Correspondence Address: JENNINGS, STROUSS & SALMON, P.L.C., 201 E. WASHINGTON ST., 11TH FLOOR, PHOENIX, AZ, 85004, US

(21) Appl. No.: 11/830,749

(22) Filed: Jul. 30, 2007

Related U.S. Application Data
(63) Continuation-in-part of application No. 11/803,050, filed on May 9, 2007.
Application No. 11/803,050 is a continuation-in-part of application No. 11/696,479, filed on Apr. 4, 2007.
(60) Provisional application No. 60/883,479, filed on Jan. 4, 2007.
(60) Provisional application No. 60/883,465, filed on Jan. 4, 2007.

(51) Int. Cl. A61K 35/60

(52) U.S. Cl. 424/523

(57) ABSTRACT

The present disclosure provides for novel krill oil-based compositions, method of administration and method of manufacture which provide for the treatment and prevention of cardiovascular disease, including the reduction of one or more significant risk factors involved with cardiovascular disease. The active ingredients of the composition include krill oil, combined in one embodiment with niacin, and combined in an alternate embodiment with polymethoxylated flavones (PMFs), and combined in yet another embodiment with Cissus quadrangularis, and combined in a further embodiment with Gynostemma pentaphyllum.

### CLAIM TO DOMESTIC PRIORITY

This Application is a continuation-in-part of and claims the benefit of priority of U.S. application Ser. No. 11/803,050 filed May 9, 2007, and U.S. application Ser. No. 11/696,479 filed on Apr. 4, 2007, which claim the benefit of priority of U.S. Application Ser. No. 60/883,479 filed Jan. 4, 2007, and U.S. Application Ser. No. 60/883,465 filed Jan. 4, 2007.

### FIELD OF THE INVENTION

This invention relates generally to the field of compositions and methods for reducing contributing factors to cardiovascular disease, namely heart attack and stroke, including reducing triglyceride levels, low density lipoproteins, total cholesterol, and blood glucose levels, among other benefits, and more specifically to a composition for reducing one or more risk factors associated with cardiovascular disease comprised of Euphausia superba (krill) oil and niacin and/or polymethoxylated flavones and/or Cissus quadrangularis and/or Gynostemma pentaphyllum (also referred to herein as Jiaogulan) that promotes healthy function of a variety of human organ and tissue systems as well as serves as a preventative agent and/or treatment for a variety of cardiovascular diseases, such as heart disease, heart attack and stroke.

### BACKGROUND OF THE INVENTION

Heart disease is the leading cause of death for all people in the United States. Stroke is the third leading cause of death. Heart disease and stroke continue to be major causes of disability and significant contributors to increases in health care costs in the United States. Epidemiologic and statistical studies have identified a number of factors that increase the risk of heart disease and stroke.

Coronary heart disease (CHD) accounts for the largest proportion of heart disease. About 12 million people in the United States have CHD. High blood cholesterol is a major risk factor for CHD that can be modified. More than 50 million U.S. adults have blood cholesterol levels that require medical advice and treatment. More than 90 million adults have cholesterol levels that are higher than desirable.

About 4 million persons have cerebrovascular disease, a major form of which is stroke. About 600,000 strokes occur each year in the United States, resulting in about 158,000 deaths. High blood pressure is known as the “silent killer” and remains a major risk factor for CHD, stroke, and heart failure. About 50 million adults in the United States have high blood pressure.

In the 1980s and 1990s, heart failure emerged as a major chronic disease for older adults. Almost 75 percent of the nearly 5 million patients with heart failure in the United States are older than 65 years. Hospitalization rates for heart failure continue to increase significantly in those aged 65 years and older.

Atrial fibrillation (AF) affects close to 2 million people in the United States. The number of existing cases of AF increases with age and is more common in males than in females. About 15 percent of strokes occur in persons with AF. Cases of AF may continue to rise as persons live longer and as more persons survive a first heart attack.

Diabetes has also been shown to increase the risk for heart attack or stroke. To that end, controlling and reducing blood glucose levels may also reduce the risk of heart attack and/or stroke. Prevention also centers on the modifiable risk factors, which include decreasing triglyceride and cholesterol levels, addressing obesity and hypertension, avoiding a sedentary lifestyle, making healthy dietary choices, and stopping smoking. A diet rich in omega-3 fatty acids and vitamin C is also recommended.

An increasingly growing number of other physiological markers and homeostatic mechanisms are currently under scientific investigation. Among these markers are low density lipoprotein and asymmetric dimethylarginine. Patients with CHD and those trying to prevent CHD are advised to reduce production of low density lipoproteins (LDLs) while increasing high density lipoproteins (HDLs), to keep blood pressure normal, to exercise and to stop smoking. These measures limit the progression of the disease. Recent studies have shown that dramatic reduction in LDL levels can cause mild regression of coronary heart disease.

Therefore, a need exists for a composition and method that assists patients in reducing total cholesterol, low density lipoproteins, blood glucose levels, triglycerides, and other factors contributing to heart disease and stroke, while increasing high density lipoproteins and providing recommended supplements such as omega-3 fatty acids and antioxidants.

### DETAILED DESCRIPTION OF VARIOUS EMBODIMENTS

The present disclosure provides for novel krill oil-based compositions, method of administration and method of manufacture which provide for the treatment, prevention and reduction of risk factors involved with cardiovascular disease. According to the present invention, cardiovascular disease is defined to include, but is not limited to, heart disease and stroke in humans. The active ingredients of the composition include krill oil, combined in one embodiment with niacin and combined in an alternate embodiment with polymethoxylated flavones (PMFs). According to the present invention PMFs include, but are not limited to, nobiletin and tangeretin.

The active ingredients in a further embodiment may include krill oil, niacin and PMFs. In yet a further embodiment, the active ingredients may include krill oil, one or more known statins, and niacin and/or PMFs. Known statins may include, but are not limited to, atorvastatin, fluvastatin, lovastatin, rosuvastatin, pravastatin, and simvastatin (including simvastatin/ezatimibe).

In yet a further embodiment, the active ingredients include krill oil and derivatives from Cissus quandrangularis. Still yet, the active ingredients may include any combination of the disclosed krill oil, Cissus quandrangularis derivatives, PMFs, niacin and/or statins.

In yet a further embodiment, the active ingredients include krill oil and derivatives from Gynostemma pentaphyllum, specifically including, but not limited to, gypenosides compounds derived from Gynostemma pentaphyllum. Still yet, the active ingredients may include any combination of the disclosed krill oil, Cissus quandrangularis derivatives, PMFs, niacin and/or statins with the gypenosides or other derivatives of Gynostemma pentaphyllum.

Any of the disclosed combinations may then be combined with any suitable pharmaceutical vehicle to provide the composition claimed herein. The composition is preferably administered once per day using soft gels or liquids. However, according to the present disclosure, methods of administration include tablet, capsule (hard, soft and gel caps), liquid, granulates, syrups and injectables.

The composition is manufactured using conventional methods of pharmaceutical manufacture, including excipients, and by combining the krill oil with niacin and/or PMFs accordingly into one administrable composition.

The novelty of this disclosure rests in the combined use of krill oil with one or more of the compounds disclosed herein. Krill (Euphausia superba) are shrimp-like crustaceans, primarily serving as the food source for the blue whale. Krill oil is commercially available. Analyses have demonstrated that krill oil extracted from these crustaceans contains important omega-3 fatty acids such as eicosapentaenoic acid (EPA) and docosahexaenoic acid (DHA), as well as important omega-6 fatty acids. Krill oil also has a high amount of potent antioxidants, including astaxanthin, vitamin A and vitamin E.

In one embodiment of the present disclosure, krill oil is present in a therapeutic amount ranging from 500 milligrams (mg) to 3,000 mg, with preferable dosages being 1,000 mg, in one embodiment and 2,000 mg in an alternate embodiment. Krill oil has been clinically shown to reduce total cholesterol, low density lipoproteins, and triglycerides. Krill oil has also been shown to increase high density lipoproteins (good cholesterol).

Krill oil has also been clinically shown to provide a reduction in inflammation through the reduction of C-reactive protein. C-reactive protein (CRP) is one of the acute phase proteins that increase during systemic inflammation. A growing number of studies have examined whether CRP levels can predict recurrent cardiovascular disease and stroke and death in different settings. High levels of CRP consistently predict new coronary events in patients with unstable angina and acute myocardial infarction (heart attack). Higher CRP levels also are associated with lower survival rate of these people. Recent studies also suggest that higher levels of CRP may increase the risk that an artery will reclose after it has been opened by balloon angioplasty.

High levels of CRP in the blood also predict recurrent events in patients with stroke and peripheral arterial disease. Most studies show that the higher the CRP levels, the higher the risk of developing heart attack. Scientific studies have found that the risk for heart attack in people in the upper third of CRP levels is twice that of those whose CRP is in the lower third. Recent studies also found an association between sudden cardiac death, peripheral arterial disease and CRP.

Krill oil has also been clinically shown to cause a reduction in blood glucose levels, a key to mitigating the effects of diabetes, a known risk factor for heart disease and stroke.

In one embodiment, krill oil is combined with niacin (vitamin B3). According to the present disclosure, niacin is present in a therapeutic amount ranging from 100 mg to 3,000 mg, with a preferable dosage of 1,000 mg. The niacin can be in either a sustained-release or immediate-release form. Niacin increases longevity and reduces mortality in patients who have suffered a first myocardial infarction. Niacin also promotes the reduction of peripheral vascular disease and symptoms of claudication.

Niacin has been clinically shown to provide a significant reduction in triglycerides, a reduction in low density lipoproteins and total cholesterol, as well as a significant increase in high density lipoproteins (including HDL-C and HDL2). When LDLs do form, niacin promotes the growth of large, fluffy LDL particle size over the more problematic small, dense LDL particle. Niacin has also been clinically shown to reduce C-reactive protein (CRP).

Niacin has the broadest effect on the lipid profile, reducing all atherogenic apolipoprotein (apo) B and increasing all antiatherogenic apo Al-containing lipoproteins, resulting in significant reduction in atherosclerotic complications and total mortality in trials. Recent research indicates novel major target sites of action in the liver to directly inhibit diacylglycerol acyltransferase 2 (DGAT2), explaining its effect on triglycerides and apo B lipoproteins, and inhibit the HDL apo Al catabolism pathway, resulting in higher HDL levels.

Patients with elevated apo B levels have an increased risk of fatal acute myocardial infarction. Small, dense lipoprotein particles (phenotype B) may have increased susceptibility to lipid peroxidative modification, resulting in higher atherogenicity and higher risk of cardiovascular disease. Higher levels of apo B may identify patients with low levels of high-density lipoprotein cholesterol and normal plasma triglyceride and cholesterol levels who are actually at elevated risk for cardiovascular disease.

Further, niacin has been clinically shown to provide a significant reduction in lipoprotein (a) (also known as Lp-A or Lp(a)). Lp(a) is a lipoprotein that resembles LDL in composition with an abnormal protein, termed [a], attached. Approximately thirty percent of individuals with heart disease have elevated Lp(a) levels. The concentration of Lp(a) in plasma is genetically determined. The gene coding for [a] is located on chromosome 6. It is inherited in a Mendelian dominant fashion, which means that approximately 50% of children of parents with elevated Lp(a) also will have elevated Lp(a).

The exact physiologic function of Lp(a) is unclear but elevated plasma levels of Lp(a) have been shown to be an independent risk factor for coronary artery disease. It is one of the best predictors of heart attack in young men, blockage of vein grafts following coronary bypass surgery, and blockages in the carotid arteries of the neck. Lp(a) likely exerts its deleterious effects by virtue of its resemblance to plasminogen. Plasminogen is a substance produced by the body to aid in the breakdown of blood clots. High plasma Lp(a) concentrations may compete with plasminogen and thereby interfere with the body's normal clot-dissolving mechanism. The Lp(a) particle is also know to be highly susceptible to oxidation, one of the early steps in coronary artery disease. Niacin reduces the production of lipoprotein A in the liver and helps to bring down the lipoprotein A in the blood.

Finally, niacin has also been shown to reduce fibrinogen. High plasma fibrinogen concentration in adulthood is associated with elevated risk of coronary heart disease and stroke. Prospective studies in healthy men and women have shown that a single fibrinogen measurement predicts fatal and non-fatal cardiovascular events as much as sixteen years later. Fibrinogen level also predicts restenosis after angioplasty. Fibrinogen may promote, together with other haemostatic factors, atherosclerotic changes and thrombosis through effects shown in vitro on platelet aggregation, blood viscosity and foam cell formation. Fibrinogen is a cardiovascular risk factor whose reduction may result in a decrease in cardiovascular events such as heart attacks and post-angioplasty restenosis.

Analyses demonstrate that the composition of krill oil and niacin provide a sixty-five to seventy percent increase in high density lipoproteins with a daily administration of the two gram krill oil (2,000 mg krill oil) composition. A daily administration of the one gram krill oil (1,000 mg krill oil) composition results in up to a sixty percent increase in high density lipoproteins with a significant reduction in other cholesterol end points identified above. Daily administration of the one gram krill oil composition also results in a CRP reduction of forty-five to fifty percent and an Lp(a) reduction of up to twenty-five percent.

In an alternate embodiment, krill oil is combined with polymethoxylated flavones (PMFs). According to the present disclosure, PMFs are present in a therapeutic amount ranging from 100 mg to 1,000 mg, with a preferable dosage of 300 mg. PMFs have been clinically shown to provide a significant reduction in triglycerides, a reduction in low density lipoproteins and total cholesterol, as well as an increase in high density lipoproteins.

PMFs have also been clinically shown to cause a reduction in blood glucose levels, a key to mitigating the effects of diabetes, a known risk factor for heart disease and stroke. In addition to benefiting blood lipid levels, PMFs have been shown to inhibit inflammatory blood components COX-2 and prostaglandin E2. PGE-2 is a hormone-like substance that plays a role in the contraction and relaxation of smooth muscle, the dilation and constriction of blood vessels, control of blood pressure, and modulation of inflammation. PMFs have been clinically shown to reduce COX-2 enzymes (associated with inflammation) without interfering with COX-1 enzyme activity. Thus, PMFs are natural selective, rather than non-selective (such as VIOXX), non-steroidal anti-inflammatory compounds (similar to NSAIDs).

PMFs also reduce pro-inflammatory agents such as interleukin-1 alpha, interleukin-1 beta, tumor necrosis factor-alpha and interleukins 6. Additionally, PMFs reduce apolipoprotein-B (apo B) levels and suppress daicylglycerol acyltransferase (DGAT2).

Analyses demonstrate that the composition of krill oil and PMFs provide up to a sixty percent increase in high density lipoproteins with a daily administration of the two gram krill oil (2,000 mg krill oil) composition and a significant reduction in other cholesterol end points identified above. The composition also results in a CRP reduction of fifty to sixty percent. The krill oil and PMF combination are advantageous over the disclosed krill oil and niacin composition in that there is little to no risk of the side effects commonly associated with niacin. The most common side effect is called “niacin flush,” which is a burning, tingling sensation in the face and chest, and red or flushed skin. However, taking an aspirin approximately thirty minutes prior to the niacin may help reduce this symptom. Other side effects may include upset stomachs, fatigue and abnormal liver function tests.

Cissus quadrangularis (commonly known as hadjad or hadjora) is an indigenous plant native to India and Africa. Although Cissus has been used in Indian culture for hundreds of years, Cissus' potential benefits to cardiovascular health and weight loss have only been demonstrated very recently.

An eight-week study was conducted that included 123 overweight and obese subjects ranging in age from 19 to 50. The group included 92 obese (BMI>30) and 31 overweight (BMI=25-29) participants; 52.8 percent were female and 47.2 percent were male. Using a randomized, double-blind, placebo-controlled design, the obese patients were divided into three groups and the overweight subjects formed a fourth group. All participants in the test groups received two daily doses (514 mg each) of a composition comprised of Cissus quadrangularis standardized to 2.5% KetoSteroids. The groups were composed as follows: Group 1 (obese) received a placebo and no diet; Group 2 (obese) received the Cissus composition with no diet; Group 3 (obese) received the Cissus composition and a diet of 2,100-2,200 calories per day. Group 4 (overweight) received the formula and no diet.

The measure results were as follows: the mean weight reduction for Group 1 was 5.1 pounds; 14.5 pounds for Group 2; 17.8 pounds for Group 3; and 8.1 pounds for Group 4. There was a decrease in plasma total cholesterol and LDL (bad) cholesterol, while HDL (good) cholesterol increased; taken together, those changes translate to a dramatic improvement in the ratio of total cholesterol to HDL and LDL cholesterol. Those in the three treatment groups also experienced a significant decrease in triglycerides, C-reactive protein, and fasting blood glucose levels. No negative side effects were observed.

Specifically, Cissus quadrangularis decreased cholesterol levels 20-times greater than placebo; Cissus quadrangularis decreased triglycerides levels 6-times greater than placebo; Cissus quadrangularis decreased LDL cholesterol levels 29-times greater than placebo; and Cissus quadrangularis increased HDL cholesterol levels 25-times greater than placebo. Additionally, Cissus quadrangularis reduced Body Mass Index (BMI) of the participants by an average of over eleven percent (11%) and overall body weight by over fifteen pounds (15 lbs.) versus the control group (placebo). In this study, participants taking the placebo actually gained body mass and weight.

According to the present disclosure, the Cissus material derived from the plant is standardized based on percent ketosteroids. A range of two to six percent ketosteroid is preferable, with approximately 5% ketosteroids being more preferable and 5.1% ketosteroids being most preferable. According to the present disclosure, standardized Cissus is present in a therapeutic amount ranging from 100 mg to 3,000 mg, with a preferable dosage of 350 mg. Krill oil compositions containing Cissus will be preferably administered using a soft gel delivery system, including, but not limited to, chewable and flavored versions. The disclosed krill oil-Cissus composition is then administered for the prevention and treatment of obesity and heart disease.

Gynostemma pentaphyllum, also called Jiaogulan is an herbaceous vine of the family Cucurbitaceae (cucumber or gourd family) indigenous to the southern reaches of China, southern Korea and Japan. Gynostemma pentaphyllum is a traditional Chinese medicine used for a variety of conditions, including elevated cholesterol.

The pharmacological anti-hyperlipidemic and hypoglycemic effectiveness of Gynostemma pentaphyllum in the obese Zucker fatty diabetic rat model has been studied. After treatment for 4 days Gynostemma pentaphyllum 250 mg/kg reduced triglyceride (33%), total cholesterol, (13%) and low density lipoprotein cholesterol levels (33%). These effects were dose-dependent and maintained for at least 5 weeks. Chronic treatment for 3-5 weeks also reduced post-prandial hypertriglyceridemia induced by olive oil 10 mg/kg in the Zucker fatty rats but had no significant effect in lowering sucrose-induced hyperglycemia in Sprague-Dawley rats.

A novel regulation by Gynostemma of glucose levels was also observed in the Zucker fatty rat model. In a glucose tolerance test in obese and lean Zucker rats pretreatment with Gynostemma pentaphyllum 250 mg/kg demonstrated glucose levels were significantly less 2 hours post challenge (20%) in the Gynostemma pentaphyllum obese rats compared to the control group. Gynostemma pentaphyllum did not significantly reduce glucose levels at 120 min in the lean strain, in contrast to the 20% decrease seen in the obese rat. In vitro, Gynostemma pentaphyllum inhibited alpha-glucosidase activity (50% inhibition at 42.8), which compared to acarbose (50% at 53.9 μg/mL). The improvement in glucose tolerance at 120 min by Gynostemma pentaphyllum in obese Zucker fatty rats but not lean rats suggests that it may improve insulin receptor sensitivity and together with the significant reduction of hypertriglyceridemia, cholesterol and low density lipoprotein cholesterol. In other study, Jiaogulan extract (200 mg/kg) in feed or 100 mg/kg for stomach infusion for 3 days also reduced the blood glucose level in insulin-dependent diabetic mice.

Similarly, stomach infusion of gypenosides (20 mg/kg) for 7 weeks to mouse or rat on high fat diet reduced significantly the serum levels of total cholesterol (TC), low-density lipoprotein (LDL), and very low-density lipoprotein (VLDL). Specifically, Jiaogulan rats were fed with high fat diet for about 6 weeks to induce the hyperlipidemia, with the total cholesterol (TC) and triglyceride (TG) in the serum of an average of 231 mg/dl and 159.9 mg/dl, respectively. Jiaogulan gypenosides at 20 mg/kg was administered to these rats for 7 weeks, their serum TC and TG were reduced to an average of 153 mg/dl and 107.0 mg/dl, respectively. Further, the good cholesterol or high-density lipoprotein (HDL) increased, as well as the ratio of HDL/LDL. Jiaogulan is thought to increase the lipid metabolism, which includes the conversion of cholesterol to Vitamin D, bile acid, and HDL. Jiaogulan also inhibits the free fatty acids (FFA) production, which leads to triglyceride synthesis by the fat cells.

Gypenosides isolated from Gynostemma pentaphyllum are also valuable in reducing inflammation and atherosclerosis. Because increased nitric oxide (NO) plays a role in these pathological conditions, the pharmacological activity of gypenosides is due to suppression of NO synthesis. The markedly increased production of nitrite by stimulation of RAW 264.7 murine macrophages with 1 μg/mL lipopolysaccharide (LPS) for 20 hrs was dose-dependently inhibited by gypenosides. When cells were pretreated with gypenosides (for 1 hr) prior to LPS stimulation, subseouent NO production was significantly attenuated. Gypenosides (25 μg/mL) produced the same maximum inhibition of LPS-induced NO production as anunoguamdine, a standard inhibitor of NOS (nitric oxide synthase) enzymes. Suppression of NO production occurred both by direct inhibition, of the activity and expression of iNOS (inducible nitric oxide synthase).

Inhibition of iNOS protein expression appears to be at the transcriptional level, since gypenosides decreased LPS-induced NF-kappaB activity in a dose-dependent manner with significant inhibition achieved following pretreatment with 10 μg/mL gypenoside. These results suggest that gypenosides derived from G. pentaphyllum suppress NO synthesis in murine macrophages by inhibiting iNOS enzymatic activity and attenuating NF-kappaB-mediated iNOS protein expression, thereby implicating a mechanism by which gypenosides may exert their therapeutic effects. The extensive antioxidant effect of GP, discussed below, also may be valuable to the prevention and treatment of various diseases such as atherosclerosis, liver disease and inflammation.

In another study, subcutaneous injection of Jiaogulan gypenosides (50 mg/kg) inhibited the development of atherosclerosis in the experimental rats. The rate of platelet thrombosis was 34% tower, whereas 68% lower for venous thrombosis, than those of the rats in control groups. Similarly, rats receiving Jiaogulan extract (35 mg/kg) intravenously for 10-20 min showed an inhibition of platelet aggregation. It was also indicated that proper concentration or doses of Jiaogulan genocides could increase the activity of Na-K-ATPase enzyme activity in the cell membranes of human red blood cells. Further, the aqueous Jiaogulan extract was shown to increase the efferent aorta output of the rabbit heart in an in vitro system. Also, administration of Jiaogulan gypenosides (50 mg/kg) intravenously lowered the blood pressure of cats, in a dose-responsive manner, under anesthetic conditions for more than 30 minutes.

Further studies have shown that Jiaogulan can prolong the life span of cells, strengthen cellular functions and promote cell proliferation. The action of gypenosides (GP, saponins of Gynostemma pentaphyllum) as an antioxidant was studied using various models of oxidant stress in phagocytes, liver microsomes and vascular endothelial cells. The results show that GP decreased superoxide anion and hydrogen peroxide content in human neutrophils and diminished chemiluminescent oxidative burst triggered by zymosan in human monocytes and murine macrophages. An increase of lipid peroxidation induced by Fe²⁺/cysteine, ascorbate/NADPH or hydrogen peroxide in liver microsomes and vascular endothelial cells was inhibited by GP.

In another study, Jiaogulan inhibited lipid peroxidation in rat liver tissue due to the pre-exposure of carbon tetrachloride (CCL). Jiaogutan gypenosides (50 mg/kg) were shown to activate DNA replication in liver cell, thus promoting liver regeneration. It was also found that GP protected biomembranes from oxidative injury by reversing the decreased membrane fluidity of liver microsomes and mitochondria, increasing mitochondrial enzyme activity in vascular endothelial cells and decreasing intracellular lactate dehydrogenase leakage from these cells. Long term administration of Jiaogulan can also inhibit the formation of gallbladder stones and lower the cholesterol level in the blood and the bile of rat. When 5% Jiaogulan extract was administered to the rats on a high cholesterol diet (1.2% cholesterol) for 3 weeks, there was a 30% reduction in the formation of gallbladder stones. On the contrary, the rats on a high cholesterol diet but without Jiaogulan treatment manifested the signs of fatty liver and hemorrhage symptom.

In another study, normal human diploid embryonic fibroblasts in an in vitro culture system containing 200 μg of Jiaogulan showed a 15.7% increase in cell proliferation or activation with Jiaogulan treatment compared with the control. Likewise, in another study skin cells from premature-aging patients with “Werner's Syndrome” treated with Jiaogulan showed a 22.7% increase in cell longevity compared with the control.

In another study, Jiaogulan extract extended the average and maximum life span of both male and female Drosophilae. The ethanol extract of Jiaogulan, at the concentrations of 0.25%, 0.5%, and 1%, respectively all extended the Drosophilae life span in an amount comparable to the effect of Vitamin E. The 0.5% Jiaogulan extract was shown to shorten the batching period of the fertilized Drosophilae eggs, and also to prolong the life of the adult Drosophilae by slowing down the aging process. Moreover, the murine model of acute aging caused by d-galactose, can be reverted to 50% by administration of aqueous extract of Jiaogulan, as demonstrated by the increased ability of the mouse to actively escape the oppressing condition. Further, administration of a 1% concentration of Jiaogulan gypenosides can elevate the enzyme activity of superoxide dismutase (SOD), which protects the aerobic organisms against the potential effects of oxygen free radicals.

Additionally, gypenosides or the aqueous extract of Jiaogulan showed other beneficial effect. At a concentration of 200 mg/kg infused into the stomach of mice rendered them swimming for longer periods of time, even with an extra weight imposed to them. This is due to the consistently higher blood glucose level and reduced glycogen consumption in the gypenosides or Jiaogulan treated mice. Mice fed with gypenosides or Jiaogulan extract also can better endure mild hypoxic (lack of oxygen) condition under normal atmosphere. They lived longer than the control mice without Jiaogulan feeding, when subjected to mild hypoxic environment. Mice fed with 10-15% Jiaogulan extract could increase their swimming time in 12° C. water. Similarly, stomach infusion of Jiaogulan at 450 mg/kg also pro-longed the life of mice enclosed in a 42° C. environment.

According to the present disclosure, the Gynostemma material derived from the plant, including, but not limited to gypenosides is standardized based on milligrams of gypenosides present in the material. According to the present disclosure, standardized Gynostemma is present in a therapeutic amount ranging from 25 mg to 500 mg, with a preferable dosage of 150 mg combined with krill oil in amount ranging from 500 mg to 4000 mg with a preferable dosage of 500 mg. Krill oil compositions containing Gynostemma will be preferably administered using a soft gel delivery system, including, but not limited to, chewable and flavored versions. The disclosed krill oil-Gynostemma composition is then administered for the prevention and treatment of cardiovascular disease, among other conditions.

The disclosed krill oil compositions also have the benefits shown in other krill oil studies, including, but not limited to improved concentration, memory and learning; decreased pain associated with arthritis; healthy brain and nervous system function; relief of premenstrual syndrome (PMS) and painful menstrual periods. Other demonstrated benefits include mood regulation, increased energy, optimal skin health and overall improved quality of life. Further, the disclosed krill oil-Cissus composition can effectively target the specific health conditions associated with Metabolic Syndrome. Still yet, the disclosed krill oil-Gynostemma pentaphyllum composition is useful other conditions caused by free radical damage.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment, and best mode of the invention known to the applicant at this time of filing the application, have been presented and are intended for the purposes of illustration and description. It is not intended to be exhaustive nor limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed is:

1. A composition for reducing one or more risk factors associated with cardiovascular disease in humans, comprising: krill oil and Gynostemma pentaphyllum.

2. The composition of claim 1, wherein the krill oil is present in an amount in a range of approximately 500 mg to 4,000 mg.

3. The composition of claim 1, wherein the Gynostemma pentaphyllum is standardized to milligrams of gypenosides.

4. The composition of claim 1, wherein the krill oil is present in an approximate amount of 2,000 mg.

5. The composition of claim 1, wherein the krill oil is present in an approximate amount of 1,000 mg.

6. The composition of claim 3, wherein the standardized Gynostemma pentaphyllum is present in an amount containing 25 mg to 500 mg of gypenosides.

7. The composition of claim 3, wherein the standardized Gynostemma pentaphyllum is present in an approximate amount containing 150 mg of gypenosides.

8. The composition of claim 1, further including a pharmaceutically acceptable carrier.

9. The composition of claim 1, further comprising statin.

10. The composition of claim 9, wherein the statin is selected from a group consisting of atorvastatin, fluvastatin, lovastatin, rusovastatin, pravastatin and simvastatin.

11. The composition of claim 1, further comprising polymethoxylated falvones.

12. The composition of claim 1, further comprising niacin.

13. The composition of claim 11, wherein the polymethoxylated flavones are present in amount in a range of approximately 100 mg to 1,000 mg.

14. The composition of claim 12, wherein the niacin is present in amount in a range of approximately 100 mg to 3,000 mg.

15. The composition of claim 1, wherein the krill oil is derived from Euphausia superba.

16. The composition of claim 11, wherein the polymethoxylated flavones are selected from a group consisting of nobiletin and tangeretin.

17. A method for reducing one or more risk factors associated with cardiovascular disease, comprising administering a composition comprising: krill oil and Gynostemma pentaphyllum.

18. The method of claim 17, wherein the krill oil is administered in an approximate amount of 1,000 mg and the Gynostemma pentaphyllum is administered in an approximate amount containing 150 mg of gypenosides.

19. The method of claim 17, wherein the composition further includes Cissus quadrangularis.

20. A method for manufacturing a composition for reducing one or more risk factors associated with cardiovascular disease, comprising: combining krill oil and Gynostemma pentaphyllum.

21. The method of claim 20, further comprising standardizing the Gynostemma pentaphyllum to milligrams of gypenosides.
